(19) United States
(12) Patent Application Publication
(10) Pub. No.: US 2025/0295837 A1
(43) Pub. Date: Sep. 25, 2025

(54) 3D PRINTING TRANSPARENT BIOINK FORMULATION

(71) Applicant: Danmarks Tekniske Universitet, Kongens Lyngby (DK)

(72) Inventors: Johan Ulrik Lind, Kongens Lyngby (DK); Carmen Radeke, Kongens Lyngby (DK)

(21) Appl. No.: 18/873,021

(22) PCT Filed: Jun. 29, 2023
(86) PCT No.: PCT/EP2023/067883

§ 371 Date: Dec. 9, 2024

(30) Foreign Application Priority Data
Jun. 30, 2022 (EP) 22182356.0

(51) Int. Cl. A61L 27/38; A61L 27/20; A61L 27/22; A61L 27/26; B33Y 70/00; C08L 1/02; C08L 89/00

(52) U.S. Cl. CPC A61L 27/3826; A61L 27/20; A61L 27/227; A61L 27/26; B33Y 70/00; C08L 1/02; C08L 89/00; A61L 2300/236; A61L 2300/252; A61L 2300/414; A61L 2400/12

(57) ABSTRACT

Bioink comprising modified fibers of MFC with a diameter of about 100-400 nm.

The present invention relates to an optically transparent aqueous 3D-printing bioink formulation, a method for producing the bioink and the use of modified microfibrillated cellulose for preparing an optically transparent aqueous 3D-printing bioink formulation.

### FIELD

Tissue engineering is a method of growing tissues and organ models in vitro or in vivo, which is driven by an immense need for more reliable preclinical models of human organs, as well as functional tissues for transplantation. 3D bioprinting is a method within tissue engineering that enables automated fabrication of tissues and organ models in the laboratory. Bioprinting allows fabrication of complex biomaterial scaffolds, engineered tissues, and micro-physiological systems to provide natural environment of the cells, so that the cells can grow, proliferate and differentiate.

One way to produce tissue using extrusion-based 3D bioprinting relies on dispensing a biomaterial ink layer-by-layer onto to a suitable surface to obtain a scaffold onto which cells are subsequently added. Another possibility is to use a bioink comprising materials to produce a scaffold together with cells, and stack these in a layer-by-layer approach. A third approach is embedded printing, where lower viscosity cell-laden inks are deposited into a reservoir biomaterial of low yield stress, which behaves like a soft solid at rest, but fluidizes in the vicinity of a moving extrusion needle.

A core challenge in 3D bio-printing is the formulation of biomaterial inks that facilitate the formation of functional tissues from embedded cells or spheroids, while simultaneously assuring printability and shape. Bioinks comprising nanofibrils are very interesting when formulating bioinks.

In the first regard, fibrillar inks structurally mimic extracellular matrix (ECM) nanofibers derived from e.g. collagen and fibronectin, that guide cellular adhesion, migration, proliferation, differentiation, and organization in the native tissue. In the second regard, fibrillary components can be potent thixotropic agents, capable of forming viscous shear-thinning solutions or viscoelastic gels with low yield stress with ideal rheology for extrusion-based printing, at low concentrations.

Two main directions currently coexist within fibrillary bioinks: The most widespread approach relies on simply applying ECM-derived biomaterials directly as the core component of the inks. A common theme for such inks is that nanofiber polymerization and gel formation occur after deposition. Common examples are collagen inks where a post-printing temperature increase to 37° C. induces polymerization or fibrin inks where thrombin is applied to induce polymerization of fibrinogen to fibrin. Similarly, for inks based on decellularized ECM (dECM) derived from primary tissues or in the form of commercial Matrigel®, gel formation is induced by collagen fiber polymerization in response to physiological temperature. The other key direction within nanofibrillar inks relies on producing micro- or nano-fibrils prior to ink formulation and printing. In these cases, the fibrils may serve as rheological modifiers, ensuring reliable extrusion or multilayer stacking. Various types of nanofibrils have been introduced, including fibrils derived from modified hyaluronic acid, mechanically fractured electrospun polymers such as polycaprolactone (PCL), and nanofibrillar cellulose.

### BACKGROUND

EP 3 326 661 describes the preparation of muscle tissues using 3D-printing. The bioink described in EP 3 326 661 comprises 0.05-606/mL of cell, 0.1-10 w/v % of cell carrier material, 0.01-1 w/v % of viscous enhancer, 1-30 v/v % of lubricant and 0.1-10 w/v % of structural material. Methylcellulose as a structural material is mentioned.

US 2017/0368255 discloses a bioink composition comprising nanofibrillated cellulose from the bacterial cellulose pellicle with fiber diameter of between 10 and 30 nm and a crosslinking component.

WO 2016/100856 describes bioinks comprising cellulose nanofibril dispersion, which is processed through different mechanical, enzymatical and chemical steps to yield dispersion with certain morphological and rheological properties. The diameter of the microfibers used was 30 nm and length above 10 μm. The content of the fibers in the composition was up to 5-8% by weight.

Present bioinks need further improvement to meet all necessary requirements.

### SUMMARY

The present invention provides optically transparent bioinks for printing tissue and organs by 3D printing and the method for producing them.

The bioink formulation of the present invention has a storage modulus from 1 Pa to 1000 kPa, and a yield stress of at least 0.5 Pa to about 1 kPa and a yield strain of less than 1000% before any possible crosslinking.

Modified cellulose fibers that can be used for the fibers of the bioink formulation of the present invention can be obtained by functionalizing the OH groups of the glucose units, and the primary alcohol on the C6 carbon in particular. The hydrogen of the OH group can be substituted with for example —CH₂CO₂H or —COCH₃ group to obtain carboxymethylated or acetylated cellulose. Alternatively, the CH₂OH unit may be oxidized to carboxylic acids and/or aldehydes using e.g. TEMPO oxidation.

The properties of the bioink of the present invention such as rheology, shear thinning behavior, viscosity, transparency can be tuned by selecting the diameter, length, and the content of the fibers of modified microcellulose.

The diameter of the fibers can be selected to simultaneously achieve optical transparency and cell alignment along fiber orientation. For that reason, the fibers used in a bioink of the present invention have a diameter of at least 100 nm but less than about 400 nm.

The modified fibers may further be covalently modified with reactive groups for bioconjugation and crosslinking purposes, such as thiols, alkenes, alkynes, azides, acrylates, methacrylate, aldehydes, groups for Diels-Alder reactions, maleimides, alcohols etc.

Further tuning can be achieved by covalent bioconjugation or non-covalent combinations with biomolecules such as cell-adhesive peptides, peptides for enzymatic crosslinking or by protein additives, such as gelatin, collagens, fibrinogen/fibrin, fibronectins, laminins, vitronectin, perlecan, nidogen, elastin, Proteoglycans such as aggrecan, decorin, biglycan brevican, neurocan, versican, periecan, syndecans, glypicans, lumican, keratocan claustrin and Glycosaminoglycans (GAGs) such as hyaluronic acid, heparin, heparan sulfate, chondroitin sulfate, dermatan sulfate, keratan sulfate. In addition to combinations with specific ECM components a complex extracellular matrix component derived from decellularized primary tissues may be used.

Combinations with other polysaccharides such as alginates, carrageenans, agar, chitin, chitosan, locust bean gum, gum arabic, xanthan gum, gellan gums, may be applied for modification of rheology, achieving crosslinking and avoiding cell adhesion.

The bioink of the present invention may or may not contain cells and further additives such as differentiation agents, growth factors and cytokines. The cells that can be used with the bioink of the present invention are for example a stem cell, an induced stem cell, an embryonic stem cell, an adult stem cell, a hematopoietic stem cell, a mesenchymal stem cell, a cardiomyocyte, a myoblast, a myofibroblast, a cardiovascular cell, an osteoblast, an osteoclast, an adipocyte, a tenocyte, a neuroblast, a fibroblast, a glioblast, a germ cell, a hepatocyte, a renal cell, a sertoli cell, a chondrocyte, an epithelial cell, a keratinocyte, a smooth muscle cell, an endothelial cell, a pericyte, a glial cell, an astrocyte, an oligodendrocyte, a neuron, an immune cell, a T-cell, a B-cell, a dendritic cell, a hormone-secreting cell, a pancreatic islet cell, a follicle-derived cell, a cancer cell.

Cells may be added in to the bioinks in the form of individually suspended cells or in the form of spheroids, organoids.

### DETAILED DESCRIPTION

Reference will be made now to various exemplary embodiments of the invention.

The present invention provides optically transparent aqueous 3D printing bioink formulation comprising fibers of modified microfibrillated cellulose
having an average diameter of about 100-400 nm;
an average length of at least 10 μm;
the fibers being dispersed in the aqueous bioink formulation.

By the term “optically transparent formulation”, a formulation is meant which is essentially transparent to the visible light, such that the formulation having transmittance of at least 90% of the visible light.

The transmittance is determined by measuring the absorbance using a Thermo Scientific NanoDrop 2000, at a path length of 1 mm, wherein the transmittance is calculated as follows:

$$\% T = {10}^{2 - A}$$

Cellulose materials are a diverse class of materials. It includes cellulose nanocrystals (CNC) with a typical length of 100-600 nm and diameters ˜2-20 nm, and microfibrillated cellulose (MFC)/nanofibrillated cellulose (NFC) where fiber diameters range from tens to several hundreds of nanometers and lengths are generally longer than 1 μm. Terms “microfibrillated cellulose” (MFC) or “nanofibrillated cellulose” (NFC) are used interchangeable in the literature. Hereinafter term microfibrillated cellulose” (MFC) will be used also for the “nanofibrillated cellulose”.

Optically transparent bioink formulations comprising non-modified MFC or NFC have not been provided due to light diffraction by the larger fibers and aggregates. Such fibers have to be degraded or otherwise chemically treated to be made suitable for the use in the bioinks.

In one embodiment diameter of the fibers of modified microfibrillated cellulose in the formulation of the present invention is 110, 120, 130, 140, 150, 160, 170, 180, 190, 200, 210, 220, 230, 240, 250, 260, 270, 280, 290, 300, 310, 320, 330, 340, 350, 360, 370, 380, or 390 nm.

In one embodiment the average diameter of the modified microfibrillated cellulose in the formulation of the present invention is in the range of from 100-400 nm.

The average length of the fibers of modified microfibrillated cellulose in the formulation of the present invention is at least the length of the cells that are used for the particular 3D bioprinting application. The common lower range for the length of the cell is about 10 μm so that the fibers also have an average length of at least 10 μm.

The fibers may be as long 1000 μm.

The fibers in the formulation of modified microfibrillated cellulose of the present invention are dispersible in the aqueous formulation and essentially do not precipitate or phase-separate.

The content of the fibers of modified microfibrillated cellulose in the formulation of the present invention is about 0.5%, 1%, 2%, 3%, 4%, 5%, 6%, 7%, 8%, 9% or about 10%.

The bioink formulation of the present application may or may not contain cells for the application in the tissue engineering.

In one embodiment the fibers of the bioink formulation of the present invention are made of carboxymethylated microfibrillated cellulose (cMFC).

Carboxymethylated microfibrillated cellulose may be prepared by carboxymethylation of the primary OH groups of the glucose units by a mercerization followed by a substitution reaction.

Such modification can be used under the condition of the present invention to yield a transparent gel with shear-thinning rheological properties comprising fibers of carboxymethylated cellulose of an average diameter of about 100-400 nm and an average length of at least 10 μm.

The fibers of carboxymethylated microfibrillated cellulose are further readily miscible with protein biomaterials, such as gelatin and collagen, such as cell-adhesive peptides, peptides for enzymatic crosslinking or by protein additives, such as gelatin, collagens, fibrinogen/fibrin, fibronectins, laminins, vitronectin, perlecan, nidogen, elastin, Proteoglycans such as aggrecan, decorin, biglycan brevican, neurocan, versican, periecan, syndecans, glypicans, lumican, keratocan claustrin and Glycosaminoglycans (GAGs) such as hyaluronic acid, heparin, heparan sulfate, chondroitin sulfate, dermatan sulfate, keratan sulfate to create cell-adhesive composites bio-printing inks. In addition to combinations with specific ECM components a complex extracellular matrix component derived from decellularized primary tissues may be used.

Furthermore, a bioink formulation of the present invention comprising fibers of carboxymethylated cellulose of an average diameter of about 100-400 nm and an average length of at least 10 μm can further comprise further biopolymers. As further biopolymers glycosaminoglycans and polysaccharides such as alginates, carrageenans, agar etc. can be used. The fibers of the formulation of the present invention together with further biopolymers are called composites.

One of the further discovering is that the composites of the present invention can serve as anisotropic scaffolds for aligning cells. This can be used for example to align skeletal myotubes in accordance with shear-induced orientation of the embedded fibers during printing.

The present invention further relates in an aspect to a method for producing the inventive bioink by
Mechanically treat wood pulp cellulose to obtain microfibrillated cellulose;
Modify the fibers of the microfibrillated cellulose,
Dispersing the fibers of the modified microcellulose in an aqueous composition.

It should be understood that any feature and/or aspect discussed in connection with the bioink formulation and in particular the fibers according to the invention apply by analogy to this aspect.

In one embodiment the modification is carboxymethylation.

In one embodiment the aqueous composition is water or a buffer solution.

The present invention also relates in another aspect to an optically transparent aqueous 3D-printing bioink formulation obtainable by the method for producing the inventive bioink by
Mechanically treat wood pulp cellulose to obtain microfibrillated cellulose;
Modify the fibers of the microfibrillated cellulose,
Dispersing the fibers of the modified microfibrillated cellulose in an aqueous composition.

It should be understood that any feature and/or aspect discussed in connection with the method, the bioink formulation and in particular the fibers according to the invention apply by analogy to this aspect.

### BRIEF DESCRIPTION OF THE FIGURES

FIG. 1 Tailoring the degree of carboxymethylation and transparency of MFC fibers: By adjusting solvent composition and reactant concentration, transparent fibers can be generated.

FIG. 2 Size and appearance of carboxymethylated fibers analyzed via SEM; Fiber diameter decreases as transparency increases. Notably, although transparent, the fibers are not degraded into nanocrystal or tiny fibers, as diameters remain above 100 nm.

FIG. 3 Presence of smaller fibers for carboxymethylation reactions done in pure isopropyl alcohol (IPA) as indicated by Transmission Electron Microscopy (TEM): Although the majority of fibers are in the range of hundreds of nanometers, smaller fibers were also observed.

FIG. 4 Rheology and printability of carboxymethylated fibers at 1% in milliQ water: With higher degree of carboxymethylation of the rheological properties of the cMFCs are compromised.

FIG. 5 Rheology, transmittance and printability of highly carboxymethylated fibers at increasing concentration: Excellent rheological properties for 3D printing can be restored by increase concentration slightly.

FIG. 6 Rheology and 3D print of fiber-enforced, cross-linkable, alginate-cMFC composite inks.

FIG. 7 Self-alignment of skeletal muscle myofibers on cMFC: gelatin substrates: C2C12 myoblasts cultured and differentiated on cMFC: gelatin composite substrates organize and align according to fiber orientation as defined by shear stress in extrusion nozzle and programmable print path.

FIG. 8 Bioprint of cell-laden cMFC inks example: Immunostain (actin) of C2C12 myoblast printed and culture within a cMFC: gelatin composite ink.

FIG. 9 Example of use of cMFC inks as support reservoir for embedded printing.

FIG. 10 Example of embedded bioprinting with murine skeletal muscle cells.

### EXAMPLES

### Example 1: Preparation of Carboxymethylated Microfibrillated Cellulose Fibers

MFC was obtained from Norwegian spruce by Borregaard in Sarpsborg (NO) and delivered as 10% paste. MFC was dispersed using an Ultra-Turrax homogenizer with a S25N-18G-ST dispersing element. The IPA: EtOH solvent mix was prepared right before the experiment using freshly opened bottles. The day before the experiment, a 5% (w/v) NaOH (2% (w/v) in pure IPA; purchased from Sigma-Aldrich) solution was prepared in the respective solvent. The day after, 10 g of MFC pulp (1 g dry content) were homogenized for 10 min. at 10,000 rpm. The homogenized MFC was heated up to 35° C. while stirring. 12 mL of a 5% (w/v) NaOH solution (600 mg) were added to the dispersed fibers and left stirring at 35° C. for 30 min. After, the temperature was increased to 45° C. Once the temperature was reached, 4 mL of a 142.2 mg/mL MCA (monochloricacetic acid, purchased from Sigma-Aldrich) solution in the respective solvent was added and left stirring at 45° C. for 3 h. Also reactions were conducted with half amount of NaOH and MCA. Around 10 ml of a 10% (v/v) acetic acid solution was added to the fibers for neutralization and the fibers were filtered. The filtered fibers were washed 3× with methanol, followed by dialysis against deionized water for three days in a 12-14 kDa cut-off dialysis tube with 2 daily water changes. The dialyzed fibers were freeze-dried and stored at room temperature until further use.

### Example 2: Tuning of Degree of Carboxymethylation of Carboxymethylated Microfibrillated Cellulose

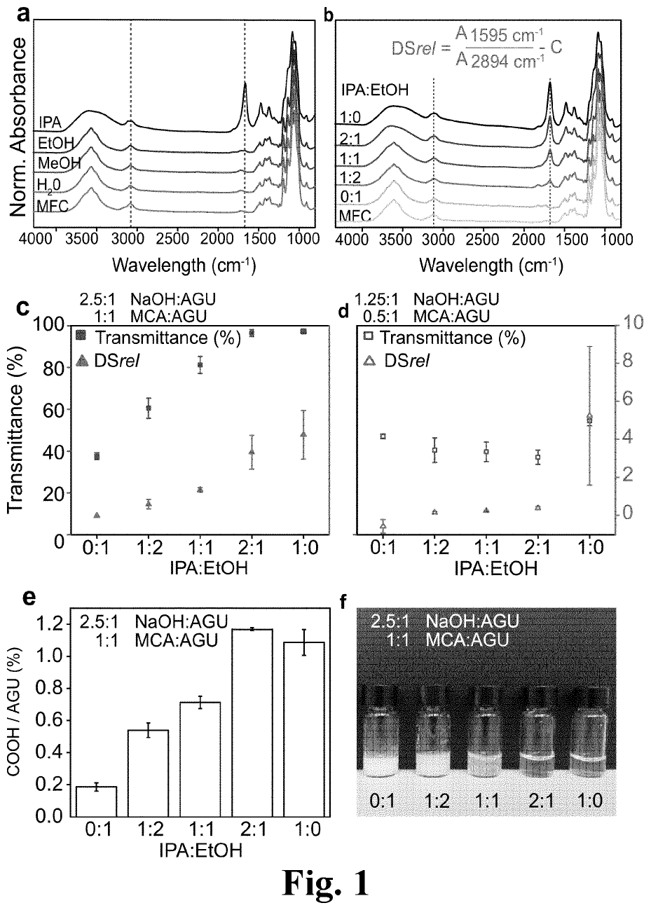

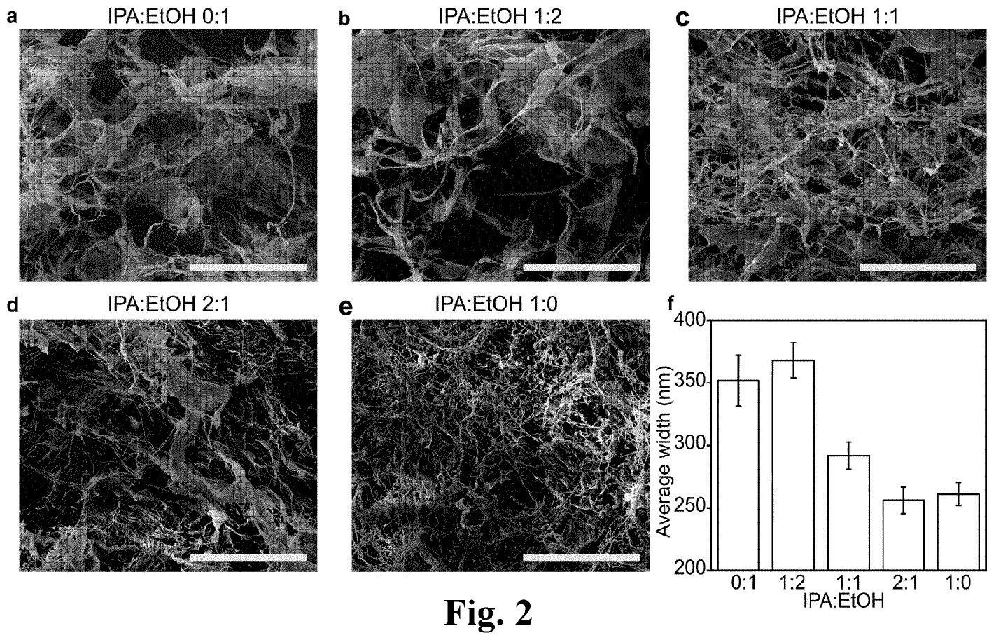

The degree of carboxymethylation may be tuned by adjusting the polarity of EtOH: IPA mixtures, which was indicated by FT-IR (FIG. 1b).

In order to create optically transparent fibers, comparison of the reaction degree and transparency fibers reacted in various IPA: EtOH mixes using two different sets of reactant concentrations. Relative to the Anhydroglucose (AGU) units these were: 2.5:1 NaOH: AGU/1:1 MCA: AGU (FIG. 1c) and 1.25:1 NaOH: AGU/0.5:1 MCA: AGU (FIG. 1d).

A more quantitative analysis of the degree of substitution was determined via titration and shows similar results as the FT-IR analysis: the amount of COOH per AGU unit increases with decreasing polarity of the solvent (FIG. 1e). Similarly, the transmittance increases with decreasing polarity of the solvent for the first set of reactants, due to an increased translucency of the fibers dispersed in aqueous solution (FIG. 1d).

For the second set of lower concentration reactants, on the other hand, the DSrel as well as the transmittance, was negligible for all solvent mixtures except pure IPA. However, for this condition we observed a large variance in the degree of reaction. This could be due to a larger sensitivity to external factors that were not controlled-such as the humidity—for reactions performed in pure IPA. The conclusion that can be made from the above data is that the degree of reaction can be tuned by changing the polarity of the solvent for reactant concentration of at least 2.5:1 NaOH: AGU and 1:1 MCA: AGU. Most importantly, that these conditions generate highly transparent fibers in EtOH: IPA solvent mixtures containing at least 66% v/v.

The increasing transparency of the fibers indicates a breakdown of the fibrillar network and better dispersion of the fibers. To evaluate if transparent fibers are successfully achieved samples that maintained physiologically relevant sizes, the fibers produced using the first EtOH: IPA reaction series using electron microscopy, were analyzed (see FIG. 2a-f).

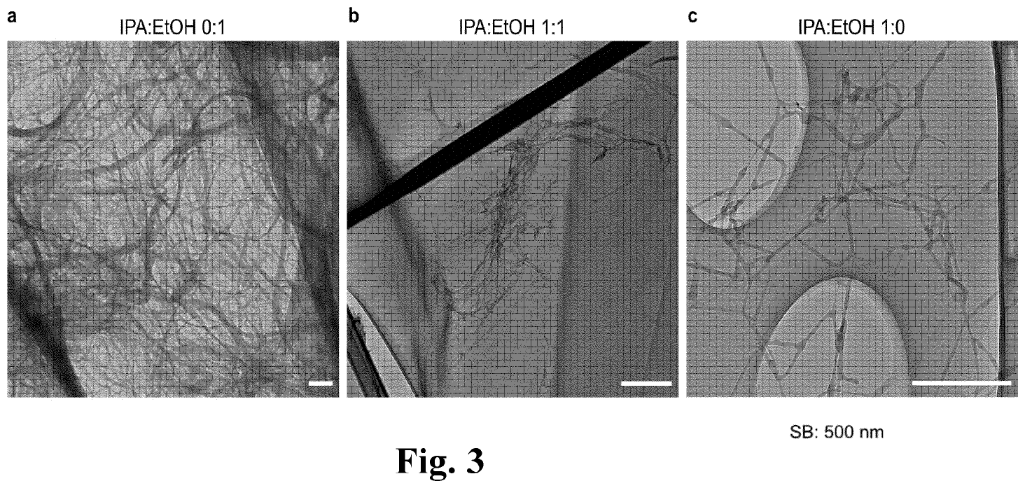

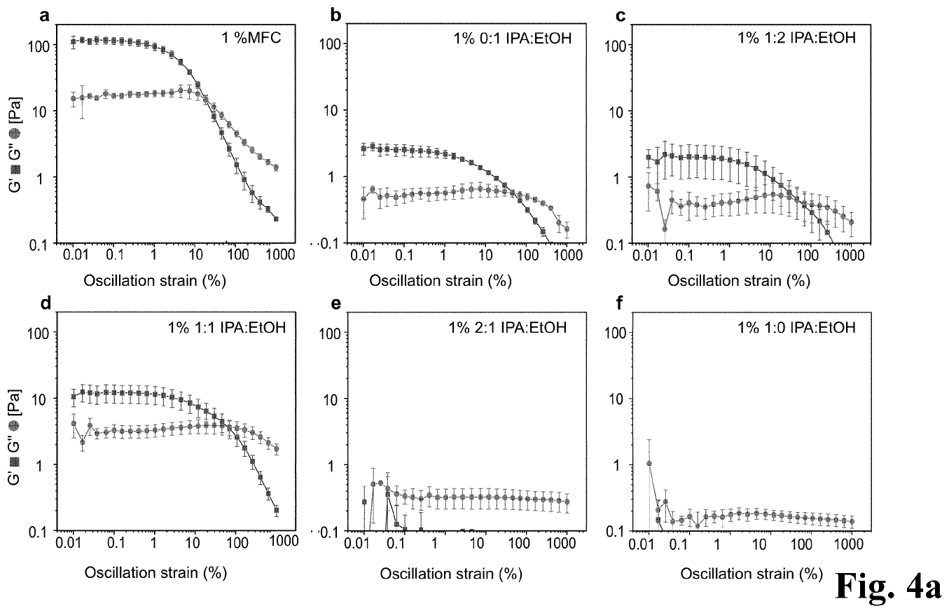

The SEM analysis showed that the average fiber diameter decreased from roughly 350 nm to 250 nm when increasing the IPA content (FIG. 2f), while maintaining lengths of tens of microns or more. While these fibers dominated, smaller structures could also be identified in TEM, especially for the reactions in pure IPA (FIG. 3).

### Example 3: Preparation of Maleimide Functionalized Carboxymethylated Microfibrillated Cellulose

100 mg of cMFC20 (obtained by performing carboxymethylation with 20% more NaOH and MCA to yield 3:1 NaOH: AGU and 1.2:1 MCA: AGU) obtained according to Example 1 and 2 polymer was transferred in a round bottom flask and dissolved in MES buffer (100 mM, pH 5.5) at a concentration of 1% w/v. The mixture was stirred until the polymer was fully dissolved. Next, 0.14 mmol of DMTMM ((4-(4,6-dimethoxy-1,3,5-triazin-2-yl)-4-methyl-morpholinium chloride)) was added and then 0.14 mmol of 2-aminoethyl maleimide. The reaction mixture was stirred at room temperature overnight for 18 h. The reaction mixture was then dialyzed against 150 mM NaCl solution for 2 days, followed by dialysis against deionized water (cutoff 10-14 kDa). Finally, the material was freeze-dried and analyzed via FTIR and H-NMR.

For FTIR analysis the samples were scanned in the range from 400 to 4000 cm−1, with 4 scans per sample cycle and a resolution of 4 cm−1. The spectra showed at 1707 cm−1 the carbonyl from maleimide, at 1654 (—C═O—) and at 1541 cm−1 the (—N—H—) amide peaks and at 695 cm−1 —C—H— bending from maleimide functionality.

The H-NMR spectrum was recorded at 500 MHz in D₂O. The H-NMR showed peaks from 3.15-4.70 ppm belonging to the protons from the cellulose backbone and a peak at 6.9 ppm from the protons from the maleimide moiety. The chemical shifts were in delta in parts per million (ppm) and were referenced against the residual solvent peak (4.79 ppm).

### Example 4: Preparation of Alcohol Functionalized Carboxymethylated Microfibrillated Cellulose

100 mg of cMFC20 (obtained by performing carboxymethylation with 20% more NaOH and MCA to yield 3:1 NaOH: AGU and 1.2:1 MCA: AGU) obtained according to Example 1 and 2 polymer was transferred in a round bottom flask and dissolved in MES buffer (100 mM, pH 5.5) at a concentration of 1% w/v. The mixture was stirred until the polymer was fully dissolved. Next, 0.42 mmol of DMTMM was added and then 0.42 mmol of 2-aminopropanol. The reaction mixture was stirred at RT overnight for 18 h. The reaction mixture was then dialyzed against 150 mM NaCl solution for 2 days, followed by dialysis against deionized water (cutoff 10-14 kDa). Finally, the material was freeze-dried and analyzed (FTIR and H-NMR).

For FTIR analysis the samples were scanned in the range from 400 to 4000 cm−1, with 4 scans per sample cycle and a resolution of 4 cm−1. The spectra showed at 1646 cm−1 the (—C═O) carbonyl from the newly formed amide bond, while-N—H-bending was not clearly visible as it overlapped with the carbonyl stretch from the starting material.

The H-NMR spectrum was recorded at 500 MHz in D₂O. The H-NMR showed peaks from 3.15-4.70 ppm belonging to the protons from the cellulose backbone and a peak at 1.3 ppm being from the protons (—CH₃) from 2-aminopropanol. The chemical shifts were in delta in parts per million (ppm) and were referenced against the residual solvent peak (4.79 ppm).

### Example 5: Preparation of Bioink Formulation Comprising Carboxymethylated Microfibrillated Cellulose

For the bioink formulation of the present invention fiber samples obtained in Example 1, when performing the carboxymethylation in 2:1 IPA: EtOH, were used. The carboxymethylated microfibers of the present invention were hereinafter referred to as cMFC.

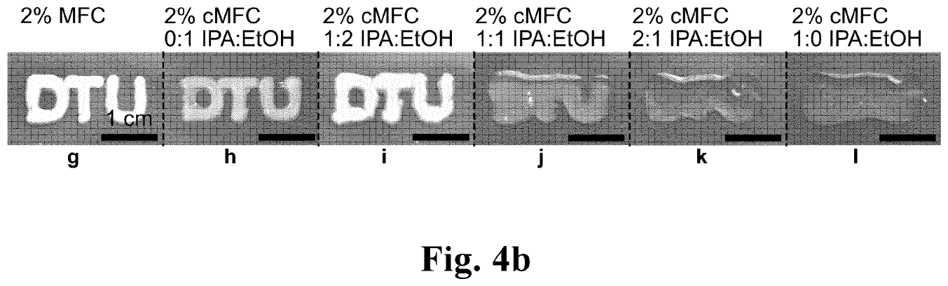

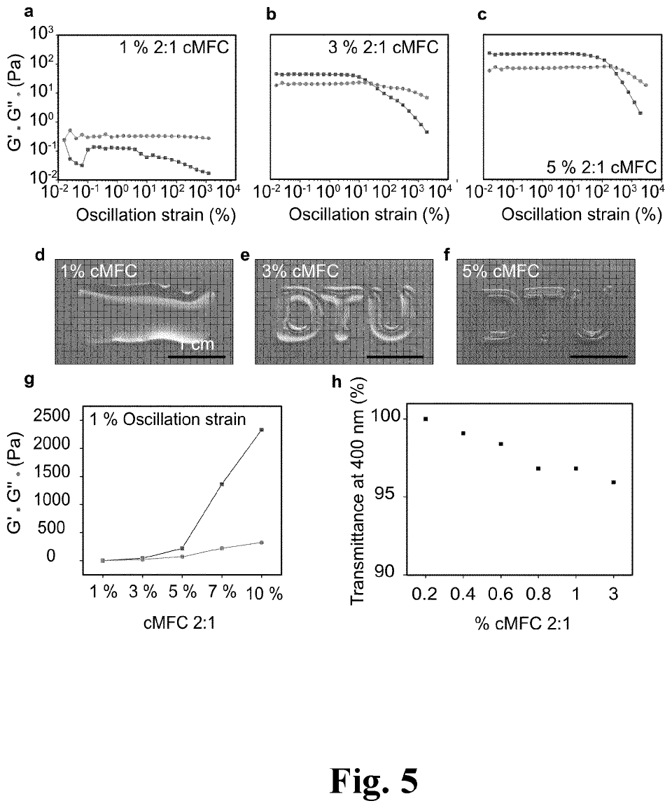

While the cMFC preserved lengths of tens of micrometers, the rheological properties were affected notably, see FIG. 4a, a-f. For the samples with a low degree of reaction, shear-thinning gels with a defined yield stress were maintained for 1% w/v samples. However, for transparent samples with a higher degree of reaction, these beneficial properties for 3D printing were largely lost. This can be seen during the printing procedure of the different cMFCs at 2% w/v (FIG. 4b, g-l). The printed structures start to flow at highly reacted, transparent samples (FIG. 4b, j, k, l). However, the rheological properties required for 3D printing can be recovered by adjusting fiber concentration. (FIG. 5a-c). By increasing the concentration up to 3% w/v the transparent fibers obtained by carboxymethylation in 2:1 IPA: EtOH, shear thinning gels were re-established. Moreover, the storage modulus and yield stress of the shear-thinning gels could readily be increased by further increasing cMFC concentration to 5% w/v. (FIG. 5c). As expected, these rheological improvements are immediately reflected in excellent 3D printing properties (FIG. 5d-f). Importantly, while increasing the fiber concentration leads to some decrease in sample transparency (FIG. 5g), the transmittance of the bioink does not fall below 95% up to 3% w/v fiber solution, (FIG. 5h). Therefore, it is possible to recover printability and shape fidelity of the fiber dispersion, by increasing its concentration, without losing transparency.

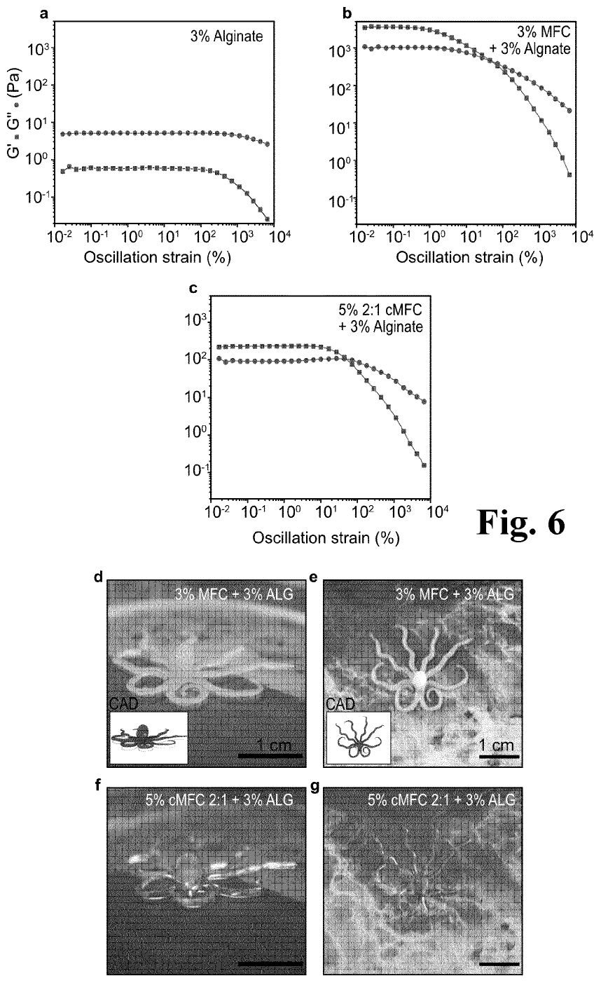

The excellent shape fidelity at higher cMFC concentrations makes possible to develop cross-linkable, transparent inks for complex structures. To demonstrate this, composite inks composed of transparent carboxymethylated fibers and alginate were formulated. MFC: alginate composite inks have been studied extensively for bioprinting, yet for native MFC fibers these composites have very limited transparency. The use of fibers of the present invention during the printing procedure highly improves the rheological properties of alginate (FIG. 6a-c).

An octopus was designed using CAD and printed with a MFC: alginate composite ink (FIGS. 6d & 6e) and cMFC: alginate (FIGS. 6f & 6g). The shape fidelity of the printed octopus was maintained after printing, yielding a complex, transparent, cross-linkable 3D structure. Still, the cMFC: alginate as ECM mimicking bioink completely lacks the protein landscape of native ECM, and most importantly cell adhesion.

A simple cMFC composite for bio-printing purposes cMFC: gelatin inks were formulated. Low concentrations of gelatin do not gel at low concentrations and behave like a viscous fluid at room temperature. Therefore, it is challenging to create a homogenous dispersion of cells within the ink due to sedimentation. The use of fibers as a composite ink with gelatin might additionally serve as guidance for cellular adhesion due to alignment of cellulose fibers during printing.

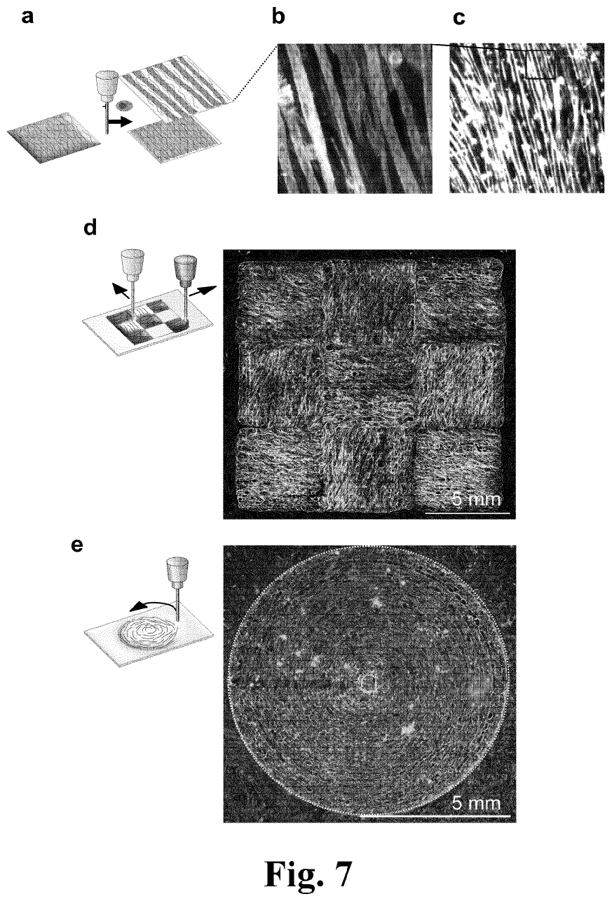

Murine and human skeletal myoblasts seeded on a cMFC: gelatin composite ink show local, parallel alignment to the print direction (FIG. 7). The alignment of myoblasts and formation of large myotubes in a size range of millimeters can be further seen when printing circles or checkerboard-like structures (FIG. 7).

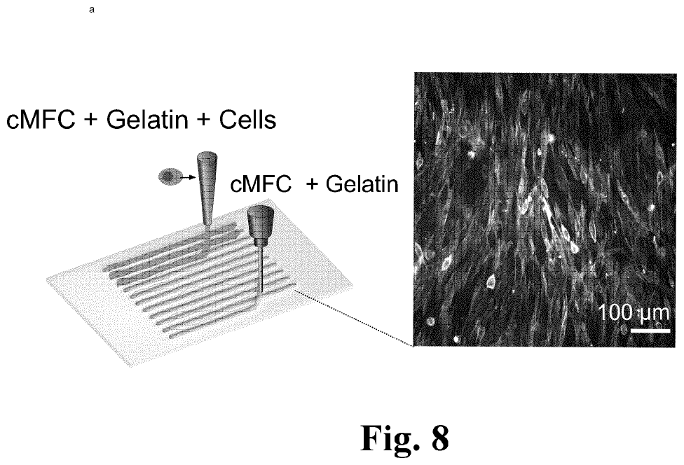

cMFCs fibers can also be applied as basis for cell-laden bioinks. For instance, bioinks based on cMFCs and gelatin were used to print C2C12 cells (FIG. 8).

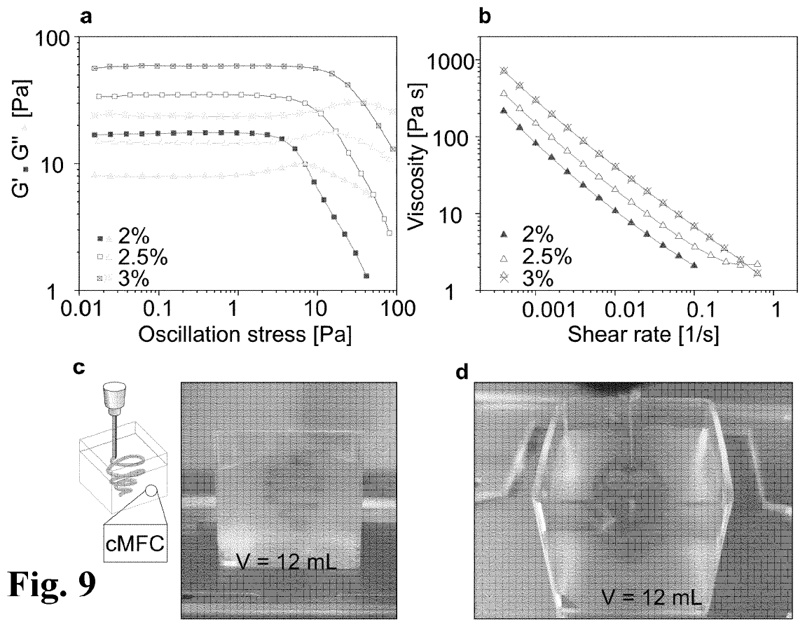

The cMFCs fibers inks can further be used as a support reservoir for embedded printing by adjusting fiber concentration to achieve a yield stress below the hydrostatic pressure of the reservoir (FIG. 9). By introducing collagen additives, a thermally annealable support can be formulated.

### Example 6-Culturing of C2C12 Murine Myoblasts and Embedded Bioprinting with Murine Skeletal Muscle Cells

### Culturing of C2C12 Murine Myoblasts

C2C12 cell-culture was performed under sterile conditions and incubated at 37° C., 100% humidity, 5% CO₂. C2C12 murine myoblasts were cultured in growth medium containing DMEM (D5796, Sigma-Aldrich), 10% fetal bovine serum (S1810, Sigma-Aldrich) and 1% P/S (P0718, Sigma-Aldrich). Cells were passaged and harvested at 80% confluency. All cells were kept within 10 passages from stock. Differentiation was initiated by changing growth medium to differentiation medium containing DMEM, 2% horse serum (H1270, Sigma-Aldrich).

Embedded Bioprinting with Murine Skeletal Muscle Cells

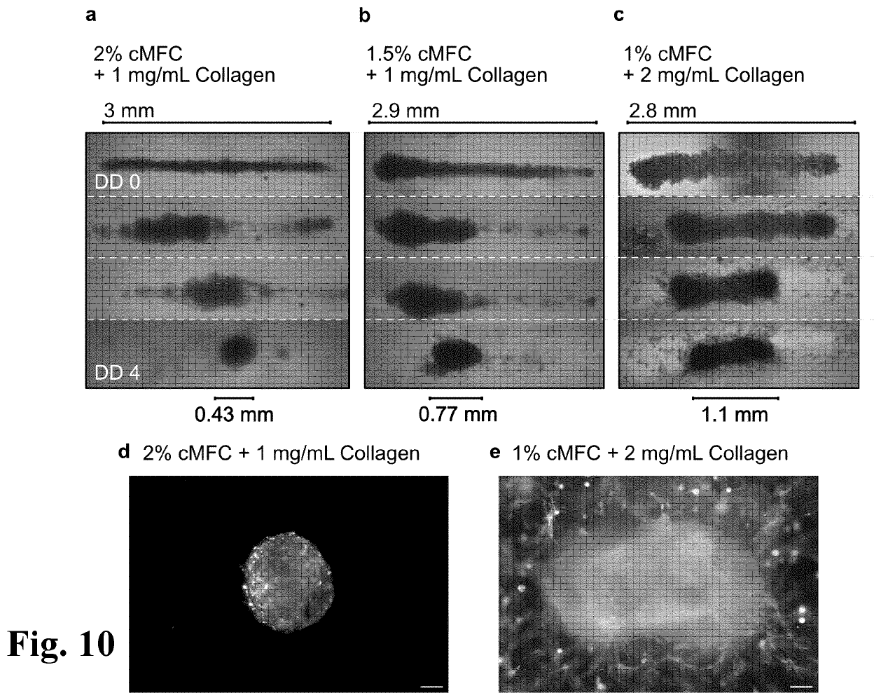

C2C12 skeletal muscle cells were cultured as described above. Bioinks as presented in FIG. 10 were composed of C2C12 cells at a concentration of 38 mio cells/mL within 10 passages. The bioinks were prepared by mixing a 1% cMFC stock 1:1 with harvested cells. A steel nozzle (ID 250 μm) was used at a feed rate of 0.5 mm/s and 0.1 μL/s extrusion rate. Right after printing, the prints were incubated for 1 h at 37° C. to ensure cross-linking of the collagen. After, growth medium prepared was added to the gels. The next day, the medium was changed to differentiation medium. Images of the tissues were taken every day.

FIG. 10 shows the compaction of C2C12 myocytes within cMFC: collagen composite gels. A 3D printed line of C2C12 myocytes results in a densely packed tissue due to compaction of the myocytes within the matrix. Low collagen content in the support matrix leads to a densely packed tissue, while higher concentrations of collagen increase cell-matrix interactions and allow for cellular migration into the support matrix. FIG. 10a shows a 2% cMFC with 1 mg/mL collagen; FIG. 10b shows 1.5% cMFC with 1 mg/mL collagen, FIG. 10c 1% cMFC with 2 mg/mL collagen. As can be seen from the before figures printed myocytes compact within 4 days of differentiation into condensed tissues. Bioink: 0.5% cMFC+38 mio/mL C2C12.

FIG. 10d shows a fluorescent stain of C2C12 myocytes in 2% cMFC with 1 mg/mL collagen, and FIG. 10e shows a 1% cMFC with 2 mg/mL collagen (Scale bar: 100 μm).

The apparatus and the method of measurement is given below:

### IR

IR spectra were recorded on a PerkinElmer Spectrum 100 FT-IR spectrometer. The spectra were recorded with a resolution of 1 cm−1 from 4000-800 cm−1. All spectra were recorded in absorbance units and normalized at 1050 cm−1. The relative degree of substitution (DSrel) was calculated by relating the intensity of the normalized absorbance (NA) of the stretching vibration of the carboxyl group (C═O) at 1595 cm−1 to the stretching vibration of the glucose backbone (C—H) at 2894 cm−1 as follows:

$${DS}_{rel} = \frac{{NA}_{1595 {cm}^{- 1}}}{{NA}_{2894 {cm}^{- 1}}} - C$$

The constant C indicates the relation between these two stretching vibrations of the carboxyl group and glucose backbone in non-oxidized cellulose.

### Titration

Between 20 and 50 mg of cMFC were weight in and transferred to a clean 100-mL Erlenmeyer flask. Calcium acetate solution 2 wt % was added to the fibers (10 ml) and let the solid material imbibe for 30 minutes. 2 drops of phenolphthalein indicator were added to the flask (prepared as 1.0 wt % in ethanol). A burette was filled with standardized NaOH solution (0.0121 M or 0.00121 M). The cMFC solution was titrated until the faint, pink endpoint is reached (persisting for at least 30 seconds). Three separate weighed samples of each cMFC sample were analyzed. The percentage of carboxyl content was calculated using the following equation:

$$Carboxyl groups [ \% ] = \frac{N \cdot V \cdot {MW}_{COOH}}{m [ mg ] \cdot 100}$$

Where N is normality of NaOH solution, V is the volume of NaOH consumed to reach the endpoint (corrected for the blank). MWCOOH is 59, corresponding to the introduced group-CH₂COOH.

### Transmittance

The absorbance of MFC and cMFC was measured using a Thermo Scientific NanoDrop 2000, at a path length of 1 mm. In general, the samples were homogenized at 1% (w/v) for 10 min. using an Ultra-Turrax homogenizer at 10,000 rpm for 10 min. The absorbance was measured right after.

Transmittance was calculated as follows:

$$\% T = {10}^{2 - A}$$

### Rheology

The rheology of each ink was analyzed using a Discovery Hybrid Rheometer (TA instruments, DE, USA) equipped with a Peltier plate thermal controller and a plate geometry with a diameter of 40 mm and a fixed gap of 1 mm. All samples were freshly prepared right before measurement. Fiber dispersion were prepared right before measurement and homogenized as described before. As a standard, amplitude sweeps were recorded at 25° C. in milliQ water at 1 Hz at an oscillation strain of 0.01-10,000%. Flow sweeps were recorded at 25° C. in PBS. Gelation curves were recorded at 1 Hz and 1%.

### SEM/TEM

Freeze-dried fibers were deposited on a carbon sticker. The samples were sputtered with a 2.4 nm gold layer. Images were recorded using a Quanta 200 FEG Cryo ESEM at an acceleration voltage of 5 KV, an aperture of 40 μm, spot size of 3.5 μm and working distance of 6 mm. Different fields of view of the same sample were analyzed at different magnifications and used for fiber counting.

### Printing of Cell-Instructive Surface and Cell Seeding

The following procedure was performed under sterile conditions: sterile solvents with 1% penicillin/streptomycin were used and cross-linking solutions were sterile filtered with a 0.45 μm pore sized filter. All syringes and needles were additionally sterilized with UV light. A composite ink consisting of 5% (w/v) low bloom gelatin and 5% (w/v) cMFC was prepared as follows:

dried fibers were suspended in DMEM at 10 k rpm for 10 minutes. Low bloom gelatin was added to the fiber suspension and heated to 45° C. for approx. 45 minutes. The solution was stirred from time to time with a spatula and shortly centrifuged to exclude air bubbles. The composite ink was printed with a steel nozzle (ID 200 μm) at a pressure of 58 kPa and feed rate of 12 mm/s. Subsequently, the print was cross-linked with a 5 U/mL mTG solution over night at 4° C. Before cell-seeding, the prints were washed 3× for 10 min. with PBS and 20 k cells/mL were added per well. Differentiation was initiated after day 3. The cells were fixed at day 7.

### Bioprinting

A cell-free surface was printed with 5% (w/v) low bloom gelatin and 5% (w/v) cMFC prepared in DMEM with 1% P/S at a feed rate of 12 mm/s, 451 kPa pneumatic printing and a 200 μm steel nozzle. Subsequently, the bioink consisting of 6% (w/v) low bloom gelatin and 2% (w/v) cMFC prepared in DMEM with 1% P/S and C2C12 myoblasts at a final concentration 4 mio/mL was printed at a feed rate of 8 mm/s, and extrusion rate of 0.5 L/s and a 450 μm diameter conical nozzle, was deposited onto the cell-instructive surface. The print was left gelling in the fridge for 5 min. Cold mTG at 10 U/mL was added and left cross-linking for 1 h at 37 deg C. The cross-linking solution was exchanged with growth medium. Differentiation was started at day 3 of cell-culture and kept for 7 days before fixation.

### Protocol for Embedded Printing:

A 2.5% (w/v) cMFC 2:1 solution was prepared in PBS and used as scaffold matrix. A 4% (w/v) alginate-ink with green colorant in PBS was prepared. The alginate-ink was printed with a 200 μm steel nozzle at 250 kPa at a feed rate of 0.3 mm/s into the scaffold matrix.

Alternatively, for 2 mL scaffold matrix, 0.4 mL collagen (5 mg/mL) was diluted in cold 0.15 mL HEPES (1 M), and 0.15 mL NaHCO₃ (37 g/L). To this, 1.3 mL cold cMFC (3%) was added and mixed carefully. The scaffold matrix was pipetted into a 24-well plate. A general bioink was prepared with 0.5% (w/v) xanthan gum and printed at 0.5 mm/s and an extrusion rate of 0.1. μL/s. The resulting embedded print was cross-linked at 37 deg C. for 20 min. The stability of the resulting gel was tested by addition of PBS to the gel. The gel did not dissolve after addition of PBS.

### Cell Staining & Imaging

Printed constructs were washed 3× with PBS. After, cells were permeabilized and fixed with 0.1% Triton X and 4% (v/v) glutaraldehyde and incubated for 20 min. at RT. The prints were washed 3× with PBS while shaking. For immunostaining, 300 μL of a 1:400 dilution of sarcomeric α-actinin monoclonal antibody was added per print and left on a shaker for 4 h. The prints were washed 3× with 0.5% BSA in PBS. Afterwards, a 1:1000 dilution of DAPI, 1:200 dilution of Alexa Fluor™ Plus 555 Phalloidin for F-actin staining and 1:400 dilution of conjugated goat anti-mouse IgG secondary antibody was added together in 0.5% BSA in PBS and incubated overnight at 4° C. The prints were washed 3× with 0.5% BSA in PBS and kept in PBS at 4° C. until further use. Images of fluorescent stains were recorded with a Nikon Eclipse Ti2 microscope and NIS-Elements software and a Zeiss Observer Z1 microscope with a mounted Zeiss AxioCam.

### Myotube Orientation Quantification

The Imagej plugin OrientationJ (REF) was used to determine the orientation of the myotubes on printed substrates. For this, F-actin stain was recorded after 7 days of differentiation. The hue and saturation of the false colored images correspond to the orientation angle and coherency, respectively. The orientation in percent was calculated over the total sum of counts.

Also disclosed are bioink formulations and methods according to any of the following items.

### Items

1) Optically transparent aqueous 3D-printing bioink formulation comprising fibers of modified microfibrillated cellulose having an average diameter of about 100-400 nm; an average length of at least 10 μm, the formulation having transmittance of at least 90% with regard to the visible light.

2) The bioink formulation of item 1, the bioink formulation having transmittance of at least 95% with regard to the visible light.

3) The bioink formulation of item 1 or 2, whereby the transmittance is determined by measuring the absorbance using a Thermo Scientific NanoDrop 2000, at a path length of 1 mm, wherein the transmittance is calculated as follows:

$$\% T = {10}^{2 - A} .$$

4) The bioink formulation of any of the preceding items, the bioink formulation having a storage modulus from 1 Pa to 1000 kPa, and a yield stress of at least 0.5 Pa to about 1 kPa and a yield strain of less than 1000%.

5) The bioink formulation of any of the preceding items, wherein the modified microfibrillated cellulose is carboxymethylated microfibrillated cellulose.

6) The bioink formulation of any of the preceding items, the bioink formulation comprising fibers having an average diameter of 110, 120, 130, 140, 150, 160, 170, 180, 190, 200, 210, 220, 230, 240, 250, 260, 270, 280, 290, 300, 310, 320 330, 340, 350, 360, 370, 380, or 390 nm; or the bioink formulation comprises fibers having an average diameter in the range of from 100-400 nm.

7) The bioink formulation of one of the preceding items, the bioink formulation having content of fibers of modified microfibrillated cellulose of about 1%, 2%, 3%, 4%, 5%, 6%, 7%, 8%, 9% or about 10%.

8) The bioink formulation according to any of the preceding items further comprising cells for engineering of tissue or organ.

9) The bioink formulation according to item 8, wherein the cells are a stem cell, an induced stem cell, an embryonic stem cell, an adult stem cell, a hematopoietic stem cell, a mesenchymal stem cell, a cardiomyocyte, a myoblast, a myofibroblast, a cardiovascular cell, an osteoblast, an osteoclast, an adipocyte, a tenocyte, a neuroblast, a fibroblast, a glioblast, a germ cell, a hepatocyte, a renal cell, a sertoli cell, a chondrocyte, an epithelial cell, a keratinocyte, a smooth muscle cell, an endothelial cell, a pericyte, a glial cell, an astrocyte, an oligodendrocyte, a neuron, an immune cell, a T-cell, a B-cell, a dendritic cell, a hormone-secreting cell, a pancreatic islet cell, a follicle-derived cell, a cancerous cell.

10) The bioink formulation according to item 8 or 9, wherein the formulation further comprises differentiation agent, growth factors, or cytokines.

11) The bioink formulation according to any of the preceding items further comprising a protein biopolymers.

12) The bioink formulation according to item 11, wherein protein biopolymers are cell-adhesive peptides, peptides for enzymatic crosslinking or by protein additives, such as gelatin, collagens, fibrinogen/fibrin, fibronectins, laminins, vitronectin, perlecan, nidogen, elastin, Proteoglycans such as aggrecan, decorin, biglycan brevican, neurocan, versican, periecan, syndecans, glypicans, lumican, keratocan claustrin and Glycosaminoglycans (GAGs) such as hyaloronic acid, heparin, heparan sulfate, chondroitin sulfate, dermatan sulfate, keratan sulfate, a complex extracellular matrix component derived from decellularized primary tissues.

13) The bioink formulation according to any of the preceding items further comprising a polysaccharide.

14) The bioink formulation according to item 13, wherein the polysaccharides is alginates, carrageenans, agar, chitin, chitosan, locust bean gum, gum arabic, xanthan gum, and gellan gums.

15) The bioink formulation according to any preceding items, wherein the printed material exhibits anisotropic properties to serve as scaffold for cells.

16) The bioink formulation according to item 15, wherein the cells are muscle cells.

17) The bioink formulation according to item 16, wherein the muscles cells are cells of skeletal muscles.

18) The bioink formulation according to item 16, wherein the muscles cells are cardiac muscle cells.

19) The bioink formulation according to any of the preceding items, further comprising a crosslinking agent.

20) The bioink formulation according to any of the preceding items, where the bioink is used as a support reservoir for embedded printing.

21) A bioink formulation according to item 20, where bioink yield stress is below the hydrostatic pressure of the reservoir.

22) Method for producing the bioink according to item 1 by
Mechanically treat wood pulp cellulose to obtain microfibrillated cellulose;
Modify the fibers of the microfibrillated cellulose.
Dispersing the fibers of the modified microcellulose in an aqueous composition.

23) Method of item 22, wherein the modification is carboxymethylation.

24) Method of item 22 or 23, wherein the aqueous composition is water or a buffer solution.

1. An optically transparent aqueous 3D-printing bioink formulation comprising a bioink, which comprises fibers of modified microfibrillated cellulose having an average diameter of about 100-400 nm; and an average length of at least 10 μm, and, wherein the bioink has a transmittance of at least 90% with regard to visible light.

2-16. (canceled)

17. The bioink formulation of claim 1, wherein the bioink has a transmittance of at least 95% with regard to visible light.

18. The bioink formulation of claim 1, wherein the transmittance is determined by measuring absorbance using a Thermo Scientific NanoDrop 2000, at a path length of 1 mm, wherein the transmittance is calculated as follows:
$$\% T = {10}^{2 - A} .$$

19. The bioink formulation of claim 1, wherein the bioink has a storage modulus from 1 Pa to 1000 Pa, and a yield stress of at least 0.5 Pa to about 1 kPa and a yield strain of less than 1000%.

20. The bioink formulation of claim 1, wherein the modified microfibrillated cellulose is a carboxymethylated microfibrillated cellulose.

21. The bioink formulation of claim 1, wherein the bioink comprises fibers having an average diameter of 110, 120, 130, 140, 150, 160, 170, 180, 190, 200, 210, 220, 230, 240, 250, 260, 270, 280, 290, 300, 310, 320 330, 340, 350, 360, 370, 380, or 390 nm; or
the bioink comprises fibers having an average diameter in the range of from 100-400 nm.

22. The bioink formulation of claim 1, wherein the bioink has a content of fibers of modified microfibrillated cellulose of about 1%, 2%, 3%, 4%, 5%, 6%, 7%, 8%, 9% or about 10%.

23. The bioink formulation according to claim 1 further comprising a stem cell, an induced stem cell, an embryonic stem cell, an adult stem cell, a hematopoietic stem cell, a mesenchymal stem cell, a cardiomyocyte, a myoblast, a myofibroblast, a cardiovascular cell, an osteoblast, an osteoclast, an adipocyte, a tenocyte, a neuroblast, a fibroblast, a glioblast, a germ cell, a hepatocyte, a renal cell, a sertoli cell, a chondrocyte, an epithelial cell, a keratinocyte, a smooth muscle cell, an endothelial cell, a pericyte, a glial cell, an astrocyte, an oligodendrocyte, a neuron, an immune cell, a T-cell, a B-cell, a dendritic cell, a hormone-secreting cell, a pancreatic islet cell, a follicle-derived cell, or a cancerous cell.

24. The bioink formulation according to claim 23, wherein the bioink formulation further comprises a differentiation agent, growth factors, or cytokines.

25. The bioink formulation according to claim 1 further comprising a cell-adhesive peptide, a peptide for enzymatic crosslinking or by protein additives, a Proteoglycan, a Glycosaminoglycan, or an extracellular matrix component.

26. The bioink formulation according to claim 1 further comprising a polysaccharide.

27. The bioink formulation according to claim 1, wherein the printed material comprises anisotropic properties suitable for attachment of cells.

28. The bioink formulation according to claim 27, wherein the cells are muscle cells selected from skeletal muscles, or cardiac muscle cells.

29. The bioink formulation according to claim 1, wherein the bioink provides a support reservoir for embedded printing, and the bioink yield stress is below the hydrostatic pressure of the reservoir.

30. A method for producing the bioink according to claim 1 comprising:
mechanically treating wood pulp cellulose to obtain a microfibrillated cellulose;
modifying the fibers of the microfibrillated cellulose, and
dispersing the fibers of the modified microcellulose in an aqueous composition.

31. The method of claim 30, wherein the microfibrillated cellulose fibers have an average diameter of about 100-400 nm and an average length of at least 10 μm.
